(19) United States
(12) Patent Application Publication
(10) Pub. No.: US 2011/0287116 A1
(43) Pub. Date: Nov. 24, 2011

(54) USE OF JASMONATES FOR TREATING HEART FAILURE AND RELATED CARDIAC DISORDERS

(75) Inventor: Brunde Broady, New York, NY (US)

(73) Assignee: BROADY HEALTH SCIENCES LLC, Coppell, TX (US)

(21) Appl. No.: 13/110,146

(22) Filed: May 18, 2011

Related U.S. Application Data
(60) Provisional application No. 61/346,459, filed on May 19, 2010.

(51) Int. Cl. A61K 36/82; A61P 9/12; A61P 9/00; A61P 9/04; A61K 31/5575; A61K 31/7034

(52) U.S. Cl. 424/729; 514/573; 514/25

(57) ABSTRACT

A formulation comprising jasmonate for improving cardiac muscle function, particularly by modulating myocardial contractility in a subject is disclosed. Such a formulation may be used to treat a heart or cardiac related disorder.

### TECHNICAL FIELD

A formulation, composition or combination of substances comprising jasmonate for improving cardiac muscle function, particularly by modulating myocardial contractility in a subject, particularly in a mammalian subject and more particularly in a human subject is disclosed. Such a formulation may be used to treat a heart or cardiac related disorder.

### BACKGROUND

Cardiac related disorders are still the first cause of morbidity and mortality in the western world. Leading cardiac related disorders include congestive, chronic and acute heart failure. These may be due to hypertension, diabetic cardiac dysfunction or myopathy (see, for example, Allow et al., 1991, Am. J. Physiol. 260:C1165-71; Ohara et al., 1991, Diabetes 40:1560-3, Fein et al., 1994, 8:65-73; Ziegelhoffer et al., 1996, Diabetes Res. Clin. Pract. 31 Suppl:S93-103; Netticadan et al., 2001, 50:2133-8; Trost et al., 2002, Diabetes 51:1166-71; Yoshikawaa et al., 2004, Nippon Yakurigaku Zasshi 123:77-86; Zhao et al., 2006, J. Physiol. Biochem. 62:1-8; Sakata et al., 2006, Mol. Ther. 13:835-8; Vasanji et al., 2004, Mol. Cell Biochem. 261;245-9), hyperthyroidism or age-related heart failure (see, for example Assayag et al., 1997, Hypertension 29:15-21; Tate et al., 1994, Med. Sci. Sports Exerc. 26:561-7; Heyliger et al., 1989, Mol. Cell Biochem. 85:75-79).

One cause of heart failure is calcium dyshomeostasis due to in part to reduced levels of Ca²⁺ ATPase, a key enzyme responsible for regulating intracellular calcium (see, for example, Schmidt, 1998, J. Mol. Cell Cardiol. 30:1929-1937; Frank, 2003, Cardiovasc. Res. 57:20-27; Frank, 1998, Basic Res. Cardiol. 93:405-411; Pieske et al., 2002, Basic Res. Cardiol. 97 suppl. 1:163-171; Ding et al. 2008, Clin. Exp. Pharmacol. Physiol. 35:827-835; Leszek, 2007, Eur. J. Heart Fail. 9:579-586; Vangheluwe, 2006, Biochim. Biophys. Acta 1763:1216-1228). Ca²⁺ ATPase is responsible for protecting the cell from excess calcium after contraction (necessary for the relaxation of the muscle) as well as the pumping of calcium into the sarcoplasmic reticulum stores for future contractions. Thus, a dysfunction in Ca²⁺ ATPase reduces the hearts ability to relax as well as to contract.

Treatments to stimulate or increase Ca²⁺ ATPase protein expression have been studied (reviewed in Prestle et al., 2003, Curr. Med. Chem. 10:967-981). Examples include, but are not limited to, gene transfers of SERCA the gene for Ca2+ATPase (Kawase, 2008, J. Am. Coll. Cardiol. 51:1112-1119, Maier et al, 2005, Cardiovasc. Res. 67:636-646) and certain drugs such as istarixime (Khan et al., Heart Fail Rev. 14:277-87 (Epub Feb. 24, 2009); Mattera et al., 2008, Eur. J. Heart Fail. 10:990-6 (Epub Aug. 6, 2008) and Rocchetti et al., 2008, J. Pharmacol. Exp. Ther. 326:957-65, (Epub Jun. 8, 2008); taurine (see, for example, Chang et al., 2004, 27:37-48 and Zhang et al., 2009, Zhongguo Zhong Yao Za Zhi 34:328-31), Astragalus or substances derived therefrom (see, for example, Li et al., 2002, Acta. Pharmacol. Sin. 23:898-904, Xu et al., 2007, Eur. J. pharmacol. 568:203-212 (Epub Apr. 19, 2007), and phospholamban inhibitors (U.S. Pat. No. 6,265,421).

### Jasmonates

Jasmonates are a family of plant stress hormones that are found in minute quantities in edible plants and characterized by cyclopentone rings. Various uses for jasmonates have been disclosed. Examples include enhancing plant growth (U.S. Pat. No. 5,436,226), repelling insects (U.S. Pat. No. 5,118,711), treating cancer (U.S. Pat. No. 6,469,061) and treating skeletal muscle degeneration caused by malnutrition and disease (U.S. Pat. No. 6,465,021, U.S. Patent Appln. Pub. No. 201000003346), pain relief (WO 2009019693), relieving psychological stress (U.S. Patent Appln. Pub. No. 200700420567), use as a component of a sleep supplement (JP2000355545), treating dry skin (U.S. Patent Appln. Pub. No. 20110085999), treating malodors on fabrics (U.S. Patent Appln. Pub. No. 20110070181). Jasmonate has also been found to increase Ca²⁺ ATPase in cardiac sarcoplasmic reticulum (see, for example, Antipenko et al., 1997, J. Biol. Chem. 272:2852-60) and skeletal muscle (see, for example, Ioumaa et al., 2002, J. Pharmacol. Exp. Ther. 300:638-46; Starling et al, 1995, Biochem. J. 308:343-6 and Starling et al., 1994, Biochemistry 15:3023-31).

### SUMMARY

Provided is a method for modulating myocardial contractility in a subject in need thereof comprising administering an amount of at least one jasmonate and optionally at least one other substance, wherein said substance is used to modulate myocardial contractility, effective to modulate said myocardial contractility. The subject may be afflicted with a heart related disorder. In a related aspect, provided is a method for treating a heart related disorder in a subject comprising administering an amount of at least one jasmonate and optionally at least one other substance used to treat cardiac disease effective to treat said heart related disorder such as acute or congestive heart failure. In a related aspect, also provided is the use of at least one jasmonate and optionally at least one other substance, wherein said substance is used to modulate myocardial contractility and/or treat a heart related disorder, to modulate said myocardial contractility and/or treat said heart related disorder and/or prepare a medicament to modulate said myocardial contractility and/or treat a heart related disorder.

Further provided are combinations, compositions or formulations comprising at least one jasmonate and optionally at least one other substance, wherein said substance is a drug or natural substance used to treat cardiac disease. In a particular embodiment, the combination, particularly composition comprises a jasmonate, a green tea extract or derivative thereof and taurine. These combinations, formulations or compositions may be used in modulating myocardial contractility and/or for treating a heart related disorder.

### DETAILED DESCRIPTION OF SPECIFIC EMBODIMENTS

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

It must be noted that as used herein and in the appended claims, the terms “composition” and “formulation” are used interchangeably.

### Definitions

As defined herein, the term “modulate” means adjusting the rate and/or amount of myocardial contractility and/or heart related disorder, by, for example adjusting the sarcoplasmic endoplasmic reticulum, preferably by increasing Ca2+ ATPase levels and/or amount and/or rate of Ca2+ release.

As defined herein, the term “a heart related disorder” is a disorder that involves the heart or blood vessels. This term may be used interchangeably with “cardiac related disorder”.

As defined herein, the term “heart failure” is the inability of the heart to supply sufficient blood flow to meet the body's needs. This may occur when the cardiac output is low (“congestive heart failure”) or when the body's requirements for oxygen and nutrients outstrip what the heart can provide (“high output cardiac failure”). It may be “acute” (rapid, sudden onset) or “chronic” (repeat episodes with recognized symptoms).

As defined herein, the terms “treat”, “treatment” and “treating” are to be understood accordingly as embracing prophylaxis and treatment or amelioration of symptoms of disease as well as treatment of the cause of the disease.

### Jasmonates

The jasmonates used in the compositions and methods set forth above may have the formula I

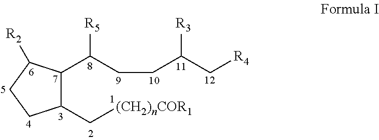

wherein:
n is 0, 1, or 2;
R₁ is OH, alkoxy, O-glucosyl, or imino,
R₂ is OH, O, alkoxy, or O-glucosyl,
R₃, R₄, and R₅ are H, OH, alkoxy or O-glucosyl, and/or wherein R₁ and R₂, or R₁ and R₄ together form a lactone, and further wherein the bonds between C₃:C₇, C₄:C₅, and C₉:C₁₀ may be double or single bonds; or a derivative of said formula, wherein the derivative has at least one of the following:
a lower acyl side chain at C₃ (free acid or ester or conjugate), a keto or hydroxy (free hydroxy or ester) moiety at the C₆ carbon, or an n-pentenyl or n-pentyl side chain at C₇,
   In a particular embodiment, the jasmonate may be jasmonate is at least one member selected from the group consisting of methyl jasmonate, jasmonic acid, jasmone, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 6-epi-cucurbic-acid lactone, 12-hydroxy-jasmonic acid, 12-hydroxy-jasmonic-acid-lactone, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, tuberonic acid-O-b-glucopyranoside, cucurbic acid-O-b-glucopyranoside 5,6-didehydrojasmonic acid, 6,7-didehydro-jasmonic acid, 7,8-didehydrojasmonic acid, cis-jasmone, methyl-dihydro-isojasmonate, dihydro-jasmone, amino acid conjugates of jasmonic acid, the lower alkyl esters of said jasmonic acids, and the carrier ligand conjugates and the sterioisomers thereof.

### Compositions

The compositions may comprise the jasmonate set forth above. Additionally, the compositions may further comprise at least one other substance used to modulate myocardial contractility and/or drug or natural substance used to treat cardiac and/or heart related disorders. This substance may include but is not limited to Astragalus or substances derived therefrom (e.g., astragaloside); gingerol; taurine; green tea or substances derived therefrom, such as a green tea extract, a catechin that may be selected from the group of catechins including, but not limited to, catechin, gallocatechiin gallate, epigallocatechin gallate, epigallocatechin, and epicatechin; Ca²⁺ sensitizers such as levosimendan, dobutamine, xamoterol; phosphodiesterase inhibitors, particularly phosphodiesterase isoenzyme III inhibitors, such as milrinone, anrinone or enoximone set forth in U.S. Pat. No. 7,279,479; Na⁺K⁺ ATPase inhibitors such as Istaroxime; (E,Z)-3-((2-aminoethoxy)imino) androstane-6,17-dione hydrochloride) digoxin, dobutamine, milrinone; phospholamban inhibitors such as those set forth in U.S. Pat. No. 6,265,421. The compositions may comprise more than one jasmonate compound and/or more than one other drug or substance.

The compositions may comprise pharmaceutically acceptable salts of the active ingredients set forth above. The phrase “pharmaceutically acceptable salts” refers to derivatives of the above disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The compositions, in particular, pharmaceutical compositions set forth above can be formulated for administration by a variety of routes including oral, transdermal, parenteral, intradermal, subcutaneous, parenteral (e.g., intravenous), intramuscular, intracardiac, intraperitoneal Such compositions are prepared in a manner well known in the pharmaceutical art and comprise as an active ingredient at least one of the compounds used in the method set forth above as described herein above ad a pharmaceutically acceptable excipients or a carrier. The amount of the active ingredient(s) in the composition is from about 0.5 to 100% per weight. The term “pharmaceutically acceptable” means approved by a regulatory agency of the Federal or a state government or listed in the U.S. Pharmacopeia or other generally recognized pharmacopeia for use in animals and, more particularly, in humans. Choosing suitable ingredients for the composition is a routine for those of ordinary skill in the art. It is evident that suitable carriers, solvents, gel forming ingredients, dispersion forming ingredients, antioxidants, colors, sweeteners, wetting compounds, release controlling components and other ingredients normally used in this field of technology may be also used. Pharmaceutical compositions in the form of intravenous solutions are preferred. The active ingredients may be formulated in the same pharmaceutical formulation. Alternatively, the active ingredients are formulated as separate pharmaceutical dosage forms. The combination of the pharmaceutical dosage forms may be packaged as a single medical product or kit for use in the method of the invention, optionally together with a package insert instructing to the correct use of the medical product.

Compositions, or particularly pharmaceutical compositions or formulations, suitable for intravenous administration of active ingredient such as injection or infusion formulation, comprise sterile isotonic solutions of the compound. Typically an intravenous infusion solution comprises from about 0.01 to 0.1 mg/ml of active ingredient. The composition or formulation may be also in the form of an intravenous infusion concentrate to be diluted with an aqueous vehicle before use. Such concentrate may comprise as a vehicle a pharmaceutically acceptable organic solvent such as dehydrated ethanol.

Another preferred embodiment is a medical product comprising, separately or together, as active ingredients one or more jasmonates or a pharmaceutically acceptable salt thereof and one or more other substances used for treating cardiac disorders as a combined preparation.

In yet another embodiment, the medical product may comprise one two or more jasmonates set forth above in separate compositions.

### Administration and Uses

As noted above, one or more jasmonate(s) optionally in combination with other substances may be used to treat heart related disorder in a subject. In particular, the subject may be a mammalian subject and even more particularly, a human subject. As noted above, in one embodiment, the disorder could, for example, be either acute or chronic heart failure, hypertension, diabetic cardiac dysfunction or myopathy or age related heart failure.

The active ingredients may be administered simultaneously, separately or sequentially. In particular, the method comprises administering to a patient an amount of active ingredients that are effective to improve the survival and/or the hemodynamic function of a subject suffering from heart failure. Preferably, the method comprises administering to a subject a synergistically effective amount of the combination. In particular, the synergistic effect makes possible to administer a low dose of the combination so as to minimize the undesirable effects of the active ingredients. The administration routes of the active ingredients include, but are not limited to, transdermal, intradermal, subcutaneous, parenteral (e.g., intravenous), intramuscular, intracardiac, intraperitoneal. In the treatment of acute heart failure, the active ingredients may be administered parenterally, and particularly, intravenously.

The compositions used may be administered e.g. intravenously using an infusion rate, which is from about 0.005 to 10 μg/kg/min, preferably from about 0.01 to 0.5 μg/kg/min, typically as low as from about 0.02 to 0.2 μg/kg/min. For an intravenous bolus a suitable dose is in the range from about 1 to 200 μg/kg, preferably from about 2 to 100 μg/kg, typically as low as from about 5 to 10 μg/kg. For the treatment of acute heart failure an intravenous bolus followed by continuous infusion may be needed.

The suggested daily dose of jasmonate(s) is in general from about 0.05 to 10 mg, preferably from 0.1 to 5 mg, more preferably from 0.2 to 2 mg, depending on the age, body weight and condition of the patient. The effective amount of jasmonate(s) to be administered to a subject depends upon the condition to be treated, the route of administration, age, weight and the condition of the patient. Similar dosages of other substances may also be used.

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various references are cited throughout this specification, each of which is incorporated herein by reference in its entirety.

What is claimed is:

1. A method for modulating myocardial contractility in a subject in need thereof comprising administering an amount of at least one jasmonate and optionally at least one other substance which is used to modulate myocardial contractility effective to modulate said myocardial contractility.

2. The method according to claim 1 wherein said subject is afflicted with a heart-related disorder.

3. The method according to claim 2 wherein said jasmonate has the structure (I) 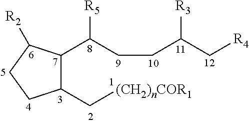
wherein:
n is b 0, 1,or 2;
R₁ is OH, alkoxy, O-glucosyl, or imino,
R₂ is OH, O, alkoxy, or O-glucosyl,
R₃, R₄, and R₅ are H, OH, alkoxy or O-glucosyl,
and/or wherein R₁ and R₂, or R₁ and R₄ together form a lactone, and further wherein the bonds between C₃:C₇, C₄:C₅, and C₉:C₁₀ may be double or single bonds; or a derivative of said formula, wherein the derivative has at least one of the following:
a lower acyl side chain at C₃ (free acid or ester or conjugate), a keto or hydroxy (free hydroxy or ester) moiety at the C₆ carbon, or an n-pentenyl or n-pentyl side chain at C₇.

4. The method according to claim 1, wherein said jasmonate is a compound selected from the group consisting of methyl jasmonate, jasmonic acid, jasmone, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 6-epi-cucurbic-acid lactone, 12-hydroxy-jasmonic acid, 12-hydroxy-jasmonic-acid-lactone, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, tuberonic acid-O-b-glucopyranoside, cucurbic acid-O-b-glucopyranoside 5,6-didehydrojasmonic acid, 6,7-didehydro-jasmonic acid, 7,8-didehydrojasmonic acid, cis-jasmone, methyl-dihydro-isojasmonate, dihydro-jasmone, amino acid conjugates of jasmonic acid, the lower alkyl esters of said jasmonic acids, and the carrier ligand conjugates and the sterioisomers thereof.

5. A method for treating a heart related disorder in a subject comprising administering an amount of at least one jasmonate and optionally at least one other substance used to treat a heart related disorder effective to treat said heart related disorder.

6. The method according to claim 5, wherein said heart related disorder is selected from the group consisting of acute heart failure, chronic heart failure, hypertension, diabetic cardiac dysfunction and age-related heart failure.

7. The method according to claim 5, wherein said other substance used to treat a heart related disorder is selected from the group consisting of a beta-agonist, gingerol, taurine, green tea or substances derived therefrom, a Na⁺K⁺ inhibitor, a Ca²⁺ sensitizer, and a phospholamban inhibitor.

8. The method according to claim 5, wherein said subject is a mammalian subject or human subject.

9. A combination comprising at least one jasmonate and at least one other substance used to treat cardiac disease.

10. The combination according to claim 9, wherein said combination is a composition.

11. The combination according to claim 9, wherein said combination comprises jasmonate, taurine and green tea extract.
